(19) United States
(12) Patent Application Publication
(10) Pub. No.: US 2010/0204626 A1
(43) Pub. Date: Aug. 12, 2010

(54) TESTICLE IMMOBILIZING DEVICE AND METHOD THEREFOR

(76) Inventor: Ron Lemond, Phoenix, AZ (US)

Correspondence Address: WEISS & MOY PC, 4204 NORTH BROWN AVENUE, SCOTTSDALE, AZ, 85251, US

(21) Appl. No.: 12/369,621

(22) Filed: Feb. 11, 2009

(51) Int. Cl. A61F 5/00

(52) U.S. Cl. 602/1

(57) ABSTRACT

A testicle immobilizing device and method therefor capable of substantially immobilizing a patient's testicles with respect to the patient's body for the purpose of facilitating examination, operation preparation, and/or operation.

### FIELD OF THE INVENTION

This invention relates generally to testicle examination or operation and, more specifically, to a testicle immobilizing device and method therefor capable of substantially immobilizing the testicles of a patient with respect to the patient's body, thereby allowing the individual examining or operating upon the patient to more easily examine or operate upon the testicles.

### BACKGROUND OF THE INVENTION

Many men will need to have their testicles examined or operated upon in their lifetime. Occasionally, the inherent nature of the male anatomy may cause difficulty in the examination of, or operation upon, the testicles, in that the testicles may have a tendency to shift or move with respect to the patient. Unfortunately, addressing this problem may require a complicated setup or may increase the steps necessary to prepare the testicles for the examination or medical procedure.

This invention addresses this problem by providing a testicle immobilizing device capable of being coupled to a patient's testicles and enabling examination of, or operation upon, the testicles without substantial movement of the testicles with respect to the patient's body. This invention enables quick and efficient examination of the testicles while adding little cost to the procedure.

### SUMMARY OF THE INVENTION

An object of the present invention is to provide a testicle immobilizing device capable of being coupled to a patient's testicles so as to substantially immobilize the patient's testicles with respect to the patient's body.

It is a further object of the present invention to provide a comfortable way for substantially immobilizing the patient's testicles during examination, preparation, or operation while keeping the patient's penis conveniently separate from the testicle examination/operation area.

### BRIEF DESCRIPTION OF THE PREFERRED EMBODIMENTS

In accordance with one embodiment of the present invention, a testicle immobilizing device is disclosed, comprising, in combination, a substantially planar member having a first end and a second end and a first edge and a second edge and a top surface and a bottom surface, the first end defining an opening between the first edge and the second edge, the opening being dimensioned to permit a scrotal sac to pass therethrough with at least one testicle being one of in contact with the top surface of the substantially planar member and above the top surface of the substantially planar member.

In accordance with another embodiment a testicle immobilizing device is disclosed, comprising, in combination, a substantially planar member being substantially U-shaped and comprised of a disposable composite material and having a first end approximately 11.5 inches from a second end and a first edge approximately 5.5 inches from a second edge and a top surface approximately 0.25 inches from a bottom surface, the first end defining an opening between the first edge and the second edge, the opening being substantially tapered and being wider at the first end and narrower proximate the second end and having, a major width proximate the first end, and a minor width proximate the second end and being between approximately 1 inch and 2 inches, the opening being dimensioned to permit a scrotal sac to pass therethrough with at least one testicle being one of in contact with the top surface of the substantially planar member and above the top surface of the substantially planar member, and the second end of the substantially planar member defining an aperture enabling placement of the substantially planar member about a hook.

In accordance with yet another embodiment, a method for immobilizing at least one testicle is disclosed, comprising, in combination, the steps of, providing a substantially planar member having a first end and a second end and a first edge and a second edge and a top surface and a bottom surface, the first end defining an opening between the first edge and the second edge, and passing a scrotal sac through the opening so that at least one testicle of the scrotal sac being one of in contact with the top surface of the substantially planar member and above the substantially planar member.

The foregoing and other objects, features, and advantages of the invention will be apparent from the following, more particular description of the preferred embodiments of the invention, as illustrated in the accompanying drawings.

### BRIEF DESCRIPTION OF THE DRAWINGS

FIG. 1 is an elevated perspective view of one embodiment of the testicle immobilizing device of the present invention in use wherein the scrotal sac is within the opening of the substantially planar member proximate the second end and the testicles are above the top surface of the planar member;

FIG. 2 is an elevated perspective view of the embodiment of the testicle immobilizing device of FIG. 1;

FIG. 3 is a bottom view of the embodiment of the testicle immobilizing device of FIG. 1, and indicates the point of view of FIG. 4;

FIG. 4 is a side, cross-sectional view of the embodiment of the testicle immobilizing device of FIG. 3, taken along line 4-4.

Common reference numerals are used throughout the drawings and detailed descriptions to indicate like elements.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

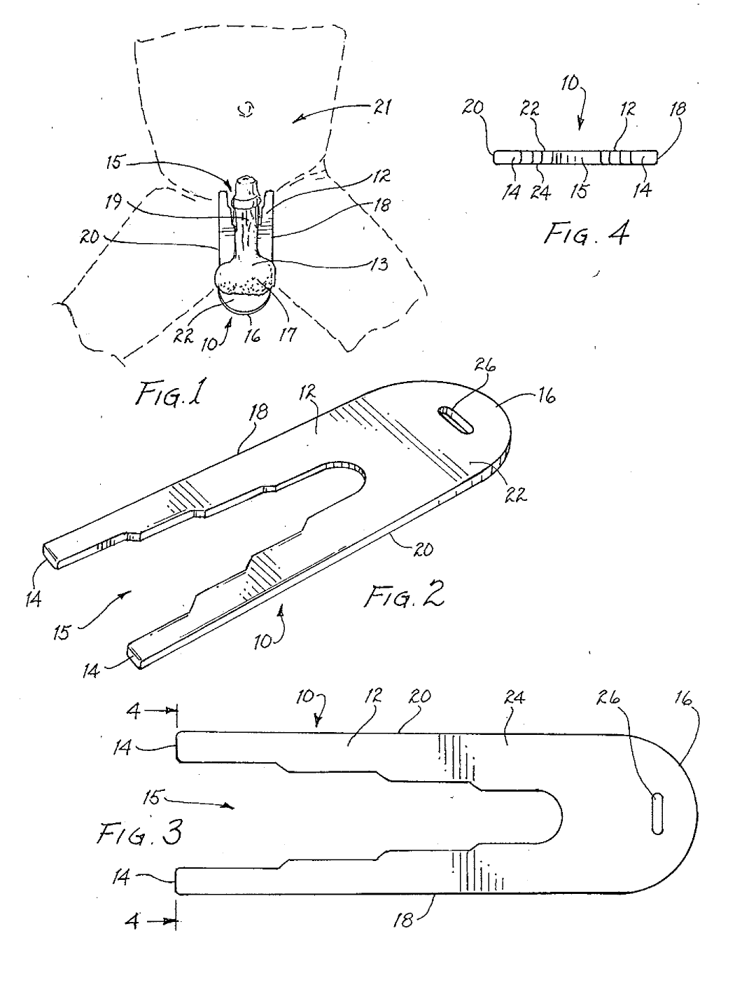

Referring now to FIGS. 1-4, a preferred embodiment of a testicle immobilizing device, referred to generically as a testicle immobilizing device 10, is disclosed. The testicle immobilizing device 10 comprises a substantially planar member 12 having a first end 14, a second end 16 (shown in FIGS. 1-3), a first edge 18, a second edge 20, a top surface 22 (shown in FIGS. 1, 2, and 4), and a bottom surface 24 (shown in FIGS. 1, 3, and 4). The first end 14 defines an opening 15 between the first edge 18 and the second edge 20, the opening 15 being dimensioned to permit a scrotal sac 13 (shown in FIG. 1) to pass therethrough with at least one testicle 17 (shown in FIG. 1) being in contact with or above a top surface 22 of the substantially planar member 12. In the preferred embodiment, the substantially planar member 12 is substantially U-shaped and comprised of a composite material, such as plastic, and is also preferably disposable, though it should be clearly understood that substantial benefit could be derived from an alternative embodiment of the present invention in which the substantially planar member 12 is of an alternative shape or comprised of an alternative material or is not disposable, so long as the substantially planar member 12 is dimensioned to permit a scrotal sac 13 to pass through an opening 15. In the preferred embodiment, the opening 15 is tapered and wider at the first end 14 and narrowing to a minor width of between approximately 1-2 inches towards the second end 16, though it should be clearly understood that substantial benefit could be derived from an alternative embodiment of the present invention in which the opening 15 is not tapered or tapered to a minor width that deviates, even substantially from the preferred minor width in either direction. In the preferred embodiment, the distance between the first edge 18 and the second edge 20 is between approximately 5-6 inches, and the distance between the first end 14 and the second end 16 is between approximately 11-12 inches, and the distance between the top surface 22 and the bottom surface 24 is between approximately 0.25-0.5 inches, though it should be clear that substantial benefit could be derived from an alternative embodiment of the present invention in which the dimensions of the substantially planar member 12 deviate, even substantially, from the preferred dimensions, in either direction.

While in the preferred embodiment, the second end 16 of the substantially planar member 12 of the testicle immobilizing device 10 defines an aperture 26 (shown in FIGS. 2 and 3), which enables the device 10 to be placed about a hook, it should be clearly understood that substantial benefit could be derived from an alternative embodiment of the present invention in which second end 16 does not define an aperture 26.

### Statement of Operation

In accordance with one embodiment of the testicle immobilizing device 10, a substantially planar member 12 is placed about a scrotal sac 13 of a patient causing the testicles 17 of the patient to come in contact with the top surface 22 of the substantially planar member 12. The patient is preferably placed in a supine position. In one embodiment of the present invention, a medical cloth or sheet of material is then placed over the penis 19 (shown in FIG. 1) of the patient while leaving the scrotal sac 13 exposed, although it should be understood that the method of the present invention may be performed without placing a medical cloth or sheet of material over the patient's penis 19 without departing from the scope and spirit of the invention. The substantially planar member 12 is then placed underneath the testicles 17 of the patient with the first end 14 being directed toward the abdomen 21 of the patient and the top surface 22 facing away from the patient. The scrotal sac 13 then passes through the opening 15 in the first end 14 and the substantially planar member 12 is moved toward the patient's abdomen 21, with the testicles being proximate the top surface 22 and the patient's body being proximate the bottom surface 24, until the scrotal sac 13 is proximate the second end 16, causing the scrotal sac 13 to be within the opening 15, between the first end 14 and second end 16, between the first edge 18 and the second edge 20, and between the top surface 22 and the bottom surface 24. Accordingly, one or more of the testicles 17 will be resting on or near the top surface 22 while the bottom surface 24 will be proximate the patient.

While the invention has been particularly shown and described with reference to preferred embodiments thereof, it will be understood by those skilled in the art that the foregoing and other changes in form and details may be made therein without departing from the spirit and scope of the invention.

I claim:

1. A testicle immobilizing device comprising:
a substantially planar member having a first end and a second end and a first edge and a second edge and a top surface and a bottom surface;
said first end defining an opening between said first edge and said second edge;
said opening being dimensioned to permit a scrotal sac to pass therethrough with at least one testicle being one of in contact with said top surface of said substantially planar member and above said top surface of said substantially planar member.

2. The testicle immobilizing device of claim 1 wherein said substantially planar member being substantially U-shaped.

3. The testicle immobilizing device of claim 1 wherein said substantially planar member being comprised of a composite material.

4. The testicle immobilizing device of claim 1 wherein said substantially planar member being disposable.

5. The testicle immobilizing device of claim 1 wherein each said at least one testicle being approximately 2 inches long and approximately 0.8 inches in breadth and approximately 1.2 inches in height.

6. The testicle immobilizing device of claim 1 wherein said opening being substantially tapered.

7. The testicle immobilizing device of claim 6 wherein said substantially tapered opening being wider at said first end and narrower proximate said second end.

8. The testicle immobilizing device of claim 7 wherein said opening having a major width proximate said first end and a minor width proximate said second end, said minor width being between approximately 1 inch and 2 inches.

9. The testicle immobilizing device of claim 8 wherein:
a distance between said first edge and said second edge being between approximately 5 inches and 6 inches;
a distance between said first end and said second end being between approximately 11 inches and 12 inches; and
a distance between said top surface and said bottom surface being between approximately 0.25 inches and 0.5 inches.

10. The testicle immobilizing device of claim 9 wherein said substantially planar member being approximately 5.5 inches by 11.5 inches by 0.25 inches.

11. The testicle immobilizing device of claim 1 wherein said second end of said substantially planar member defining an aperture.

12. The testicle immobilizing device of claim 11 wherein said aperture being dimensioned to allow placement of said substantially planar member about a hook.

13. A testicle immobilizing device comprising:
a substantially planar member being substantially U-shaped and comprised of a disposable composite material and having a first end approximately 11.5 inches from a second end and a first edge approximately 5.5 inches from a second edge and a top surface approximately 0.25 inches from a bottom surface;
said first end defining an opening between said first edge and said second edge;
said opening being substantially tapered and being wider at said first end and narrower proximate said second end and having:
a major width proximate said first end; and
a minor width proximate said second end and being between approximately 1 inch and 2 inches;
said opening being dimensioned to permit a scrotal sac to pass therethrough with at least one testicle being one of in contact with said top surface of said substantially planar member and above said top surface of said substantially planar member; and
said second end of said substantially planar member defining an aperture enabling placement of said substantially planar member about a hook.

14. A method for immobilizing at least one testicle comprising, in combination, the steps of:
providing a substantially planar member having a first end and a second end and a first edge and a second edge and a top surface and a bottom surface, said first end defining an opening between said first edge and said second edge; and
passing a scrotal sac through said opening so that at least one testicle of said scrotal sac being one of in contact with said top surface of said substantially planar member and above said substantially planar member.

15. The method of claim 14 wherein said at least one testicle being proximate said second end.

16. The method of claim 15 further comprising the steps of:
providing a sheet of material; and
placing said sheet of material over a penis proximate said at least one testicle.

17. The method of claim 16 further comprising the step of one of operating on and examining said at least one testicle.

18. The method of claim 17 wherein
said first end being proximate an abdomen wherein said abdomen being proximate said at least one testicle.
